(19) United States
(12) Patent Application Publication
(10) Pub. No.: US 2021/0393274 A1
(43) Pub. Date: Dec. 23, 2021

(54) DEVICE FOR REPEATED INTRADERMAL INJECTIONS WITHIN AN ORGANIC TISSUE

(71) Applicant: UNIVERSITÉ LAVAL, Quebec (CA)

(72) Inventors: Gary Kobinger, Quebec (CA); Jean Ruel, Quebec (CA); Alejandro Gomez, Quebec (CA); Marc-Andre PLOURDE CAMPAGNA, Quebec (CA)

(21) Appl. No.: 17/279,384

(22) PCT Filed: Sep. 10, 2019
(86) PCT No.: PCT/CA2019/051265

§ 371 Date: Mar. 24, 2021

Related U.S. Application Data
(60) Provisional application No. 62/736,865, filed on Sep. 26, 2018.

(51) Int. Cl. A61B 17/20

(52) U.S. Cl. CPC A61B 17/205

(57) ABSTRACT

A device for repeated intradermal injections within an organic tissue. The device comprises a support defining an internal housing for receiving a vial containing an aqueous solution, a source of pressurized air in fluid communication with the vial, a tube defining a pressurized solution path, a hollow injection needle, an injection head, a driven element with a distal end connected to the injection head, an actuator connected to the driven element, and a valve in fluid communication with the pressurized solution path for controlling a flow of the pressurized solution into the tube from the vial up to the distal end of the hollow injection needle.

### FIELD

The present invention relates to a device for repeated intradermal injections within an organic tissue. The device comprises a support defining an internal housing for receiving a vial containing an aqueous solution, a source of pressurized air in fluid communication with the vial, a tube defining a pressurized solution path, a hollow injection needle, an injection head, a driven element with a distal end connected to the injection head, an actuator connected to the driven element, and a valve in fluid communication with the pressurized solution path for controlling a flow of the pressurized solution into the tube from the vial up to the distal end of the hollow injection needle.

### BACKGROUND

In the last few years, new routes of vaccine administration have been studied with the objective to increase the immunogenicity of vaccines and therefore reduce the number of injections needed to generate a protective immune response against a specific pathogen. In particular, intradermal delivery of vaccines can generate broad immune responses and protect against pathogens with a lesser number of vaccine doses. The skin is a promising route for the administration of vaccines given that the dermis and epidermis are abundant in immune cells, such as antigen-presenting cells. In fact, recent data in animals and human trials demonstrate that intradermal administration is better than traditional administration of vaccines into the muscle or subcutaneous tissue. Currently, existing devices used for intradermal vaccination (ex. intradermal needle/syringes, gene gun, jet injectors, electroporation, or microneedle patches) can only deliver a small amount of vaccine preparation due to the limited skin area that can be accessed from a needle injection. In fact, the maximum volume that can be administered to humans and most large animal species is about 0.1 ml per injection.

In other injection strategies where the vaccine solution is first put on the skin surface and where oscillating needles used to penetrate the injection site (passive migration of the solution) do not control the volume of inoculated solution (vaccine). This methodology does not allow the complete administration/injection of the immunogenic solution (vaccine) into the skin and, as a result, a considerable amount of solution does not penetrate into the dermis where immune cells are present.

According to broad aspects of the invention, the device for repeated intradermal injections within an organic tissue seeks to address the limitations and drawbacks of the prior devices or injectors by providing a device that is capable of safely administering a pressurized aqueous immunogenic solution (vaccine) in an organic tissue. More particularly, in the device, during downward and upward movements of the hollow injection needle(s) into the organic tissue, a valve is adapted to allow passage of the pressurized solution into the hollow injection needle(s) for injecting the pressurized solution into the organic tissue. With the device, a volume of solution of between 0.5 ml and 1.0 ml may be injected into the organic tissue over a time duration of about 30 seconds to about 60 seconds.

### SUMMARY

As embodied and broadly described herein, according to a broad aspect, the invention provides a device for repeated intradermal injections within an organic tissue, the device comprising: a support defining an internal housing for receiving a vial containing an aqueous solution and having a cap for closing the vial, the support comprising a base comprising first and second hollow needles extending upwardly from the base, the first and second hollow needles extending along first and second longitudinal axes and each comprising proximal and distal ends, wherein, when a user inserts the vial in the housing, the cap abuts against the base and each distal end of the first and second needles pierces the cap for passing through the cap and being located in the vial; a source of pressurized air in fluid communication with the proximal end of the first hollow needle of the base for injecting pressurized air in the vial; a tube comprising proximal and distal ends, the proximal end of the tube being in fluid communication with the proximal end of the second hollow needle of the base for allowing pressurized solution to pass through the tube that defines a pressurized solution path; a hollow injection needle extending along an injection longitudinal axis, the hollow injection needle comprising proximal and distal ends; an injection head covering the proximal end of the hollow injection needle; the injection head comprising an inlet in fluid communication with the distal end of the tube and an outlet in fluid communication with the proximal end of the hollow injection needle; a driven element extending along a main longitudinal axis and comprising proximal and distal ends, the distal end of the driven element being connected to the injection head; an actuator connected to the proximal end of the driven element; and a valve in fluid communication with the pressurized solution path for controlling a flow of the pressurized solution into the tube from the vial up to the distal end of the hollow injection needle; wherein in use, the actuator moves the driven element along the main longitudinal axis at a frequency of between 80 Hz and 150 Hz and between a first position, wherein the distal end of the hollow injection needle is proximate the organic tissue, and a second position, wherein the distal end of the hollow injection needle is within the organic tissue at a depth of between 1 mm and 4 mm; and wherein, during movements of the hollow injection needle between the first and second positions, the valve is adapted to allow passage of pressurized solution into the hollow injection needle for injecting the pressurized solution into the organic tissue.

As embodied and broadly described herein, according to another broad aspect, the invention provides a device for repeated intradermal injections within an organic tissue, the device comprising: a support defining an internal housing for receiving a vial containing an aqueous solution and having a cap for closing the vial, the support comprising a base comprising first and second hollow needles extending upwardly from the base, the first and second hollow needles extending along first and second longitudinal axes and each comprising proximal and distal ends, wherein, when a user inserts the vial in the housing, the cap abuts against the base and each distal end of the first and second needles pierces the cap for passing through the cap and being located in the vial; a source of pressurized air in fluid communication with the proximal end of the first hollow needle of the base for injecting pressurized air in the vial; a tube comprising proximal and distal ends, the proximal end of the tube being in fluid communication with the proximal end of the second hollow needle of the base for allowing pressurized solution to pass through the tube that defines a pressurized solution path; a plurality of hollow injection needles, each of the hollow injection needle extending along an injection longitudinal axis and comprising proximal and distal ends; an injection head covering the proximal ends of the hollow injection needles; the injection head comprising an inlet in fluid communication with the distal end of the tube and an outlet in fluid communication with the proximal ends of the hollow injection needles; a driven element extending along a main longitudinal axis and comprising proximal and distal ends, the distal end of the driven element being connected to the injection head; an actuator connected to the proximal end of the driven element; and a valve in fluid communication with the pressurized solution path for controlling a flow of the pressurized solution into the tube from the vial up to the distal ends of the hollow injection needles; wherein in use, the actuator moves the driven element along the main longitudinal axis at a frequency of between 80 Hz and 150 Hz and between a first position, wherein the distal ends of the hollow injection needles are proximate the organic tissue, and a second position, wherein the distal ends of the hollow injection needles are within the organic tissue at a depth of between 1 mm and 4 mm; and wherein, during movements of the hollow injection needles between the first and second positions, the valve is adapted to allow passage of pressurized solution into the hollow injection needles for injecting the pressurized solution into the organic tissue.

### BRIEF DESCRIPTION OF THE DRAWINGS

A detailed description of the embodiments of the present invention is provided herein below, by way of example only, with reference to the accompanying drawings, in which:

FIG. 1 is a perspective view of the device for repeated intradermal injections within an organic tissue in accordance with a first embodiment of the invention;

FIG. 2 is a side elevational view of the device of FIG. 1;

FIG. 3 is a bottom view of the device of FIG. 1;

FIG. 4 is an enlarged perspective view of the hollow guide portion, driven element and control/injection components of the device of FIG. 1;

FIG. 5 is an enlarged perspective view of the driven element, injection head and injection needles of the device of FIG. 1;

FIG. 6 is a perspective view of the device for repeated intradermal injections within an organic tissue in accordance with a second embodiment of the invention;

FIG. 7 is a side elevational view of the device of FIG. 6;

FIG. 8 is an enlarged perspective view of the hollow guide portion, driven element and control/injection components of the device of FIG. 6;

FIG. 9 is a partially cross-sectional view of the hollow guide portion, driven element and control/injection components of the device of FIG. 6;

FIG. 10 is and enlarged fragmentary view of the injection needles of the device of FIG. 1 or of FIG. 6;

FIG. 11 is a cross-sectional view taken along line 11-11;

FIG. 12 is a perspective view of the support of the device of FIG. 1 or of FIG. 6, the support defining an internal housing for receiving a vial containing an aqueous solution and comprising a base with first and second hollow needles for piercing the cap or closure of the vial;

FIG. 13 is a side elevational view of the support of FIG. 12;

FIG. 14 is a cross-sectional view taken along line 14-14;

FIG. 15 is a cross-sectional view taken along line 15-15;

FIG. 16 is a cross-sectional view taken along line 16-16;

FIGS. 17 and 18 are diagrams schematically illustrating electric circuits and pressurized flow paths for the device of FIG. 1 or of FIG. 6;

FIG. 19 shows graphical results of immunization experiments carried out in rabbits for Ebola glycoprotein (GP) specific IgG;

FIG. 20 shows graphical results of immunization experiments carried out in mice;

FIGS. 21A and 21B show graphical results of immunization experiments carried out in guinea pigs; and

FIG. 22 shows graphical results of immunization experiments carried out in non-human primates.

In the drawings, embodiments of the invention are illustrated by way of examples. It is to be expressly understood that the description and drawings are only for the purpose of illustration and are an aid for understanding. They are not intended to be a definition of the limits of the invention.

### DETAILED DESCRIPTION OF EMBODIMENTS

Before any variants, examples or preferred embodiments of the invention are explained in detail, it is to be understood that the invention is not limited in its application to the details of construction and the arrangement of components set forth in the following description or illustrated in the drawings. The invention is capable of other variants or embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional suitable items. Unless specified or limited otherwise, the terms “mounted,” “connected,” “supported,” and “coupled” and variations thereof are used broadly and encompass both direct and indirect mountings, connections, supports, and couplings and are thus intended to include direct connections between two members without any other members interposed therebetween and indirect connections between members in which one or more other members are interposed therebetween. Further, “connected” and “coupled” are not restricted to physical or mechanical connections or couplings. Additionally, the words “lower”, “upper”, “upward”, “down” and “downward” designate directions in the drawings to which reference is made. Similarly, the words “left”, “right”, “front” and “rear” designate locations or positions in the drawings to which reference is made. The terminology includes the words specifically mentioned above, derivatives thereof, and words or similar import.

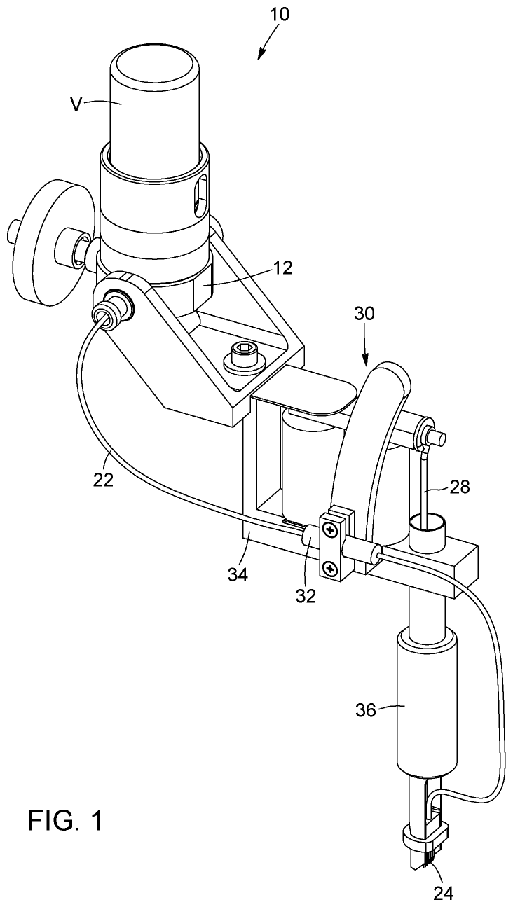

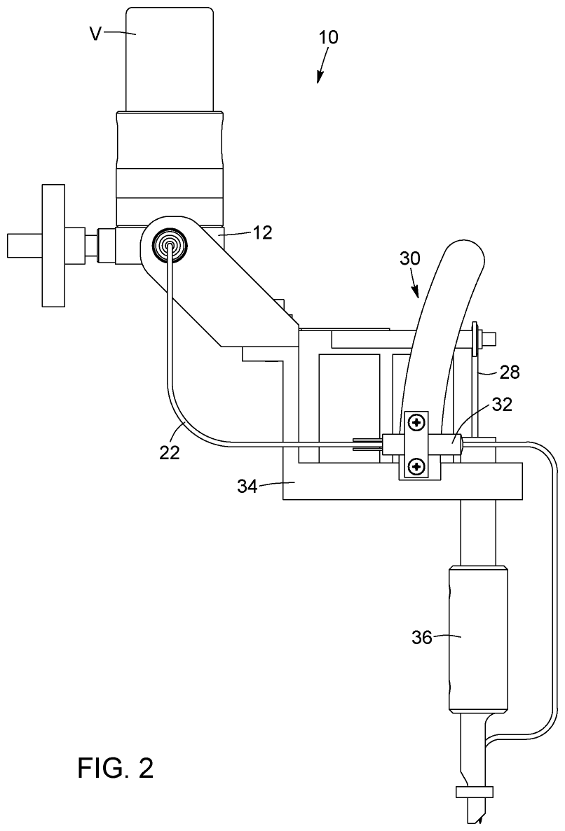

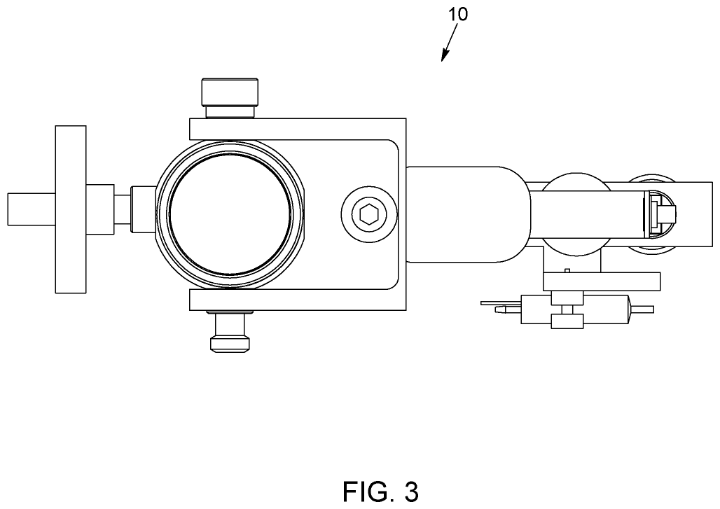

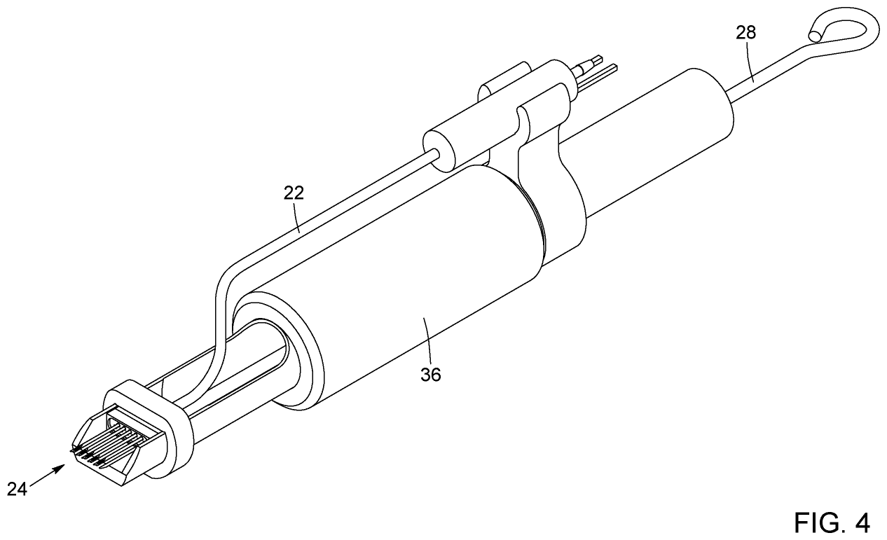

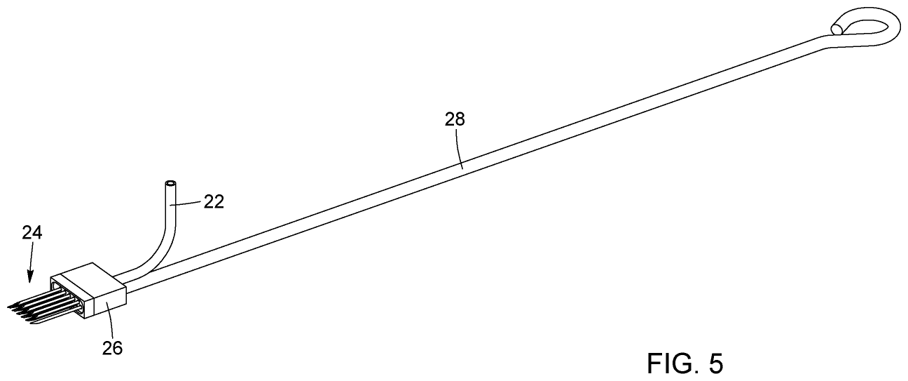

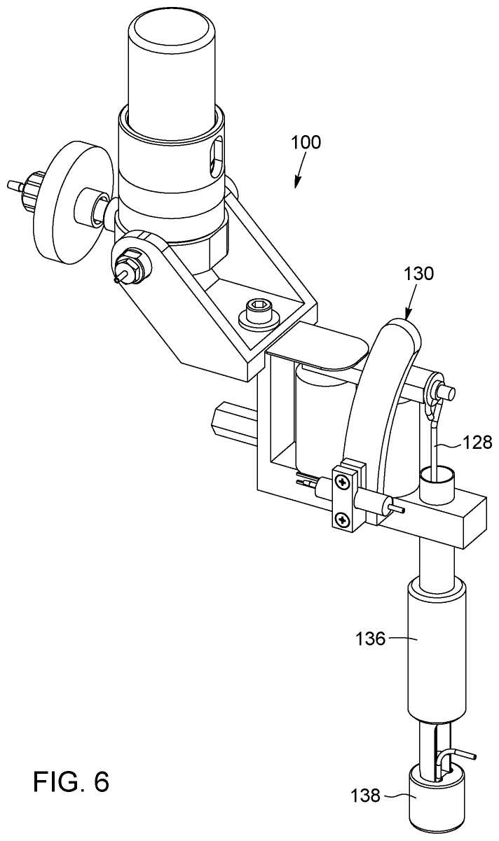

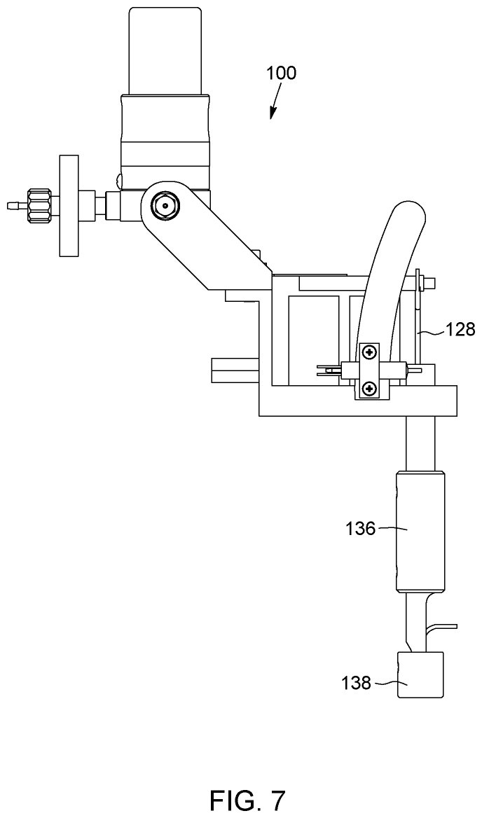

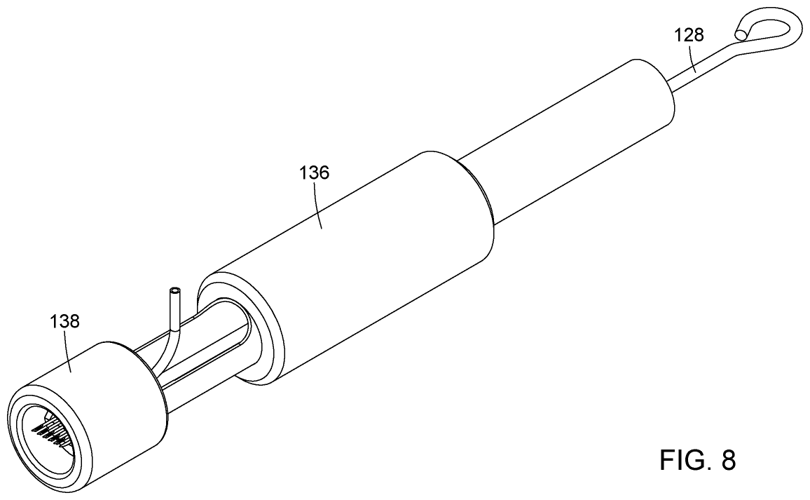

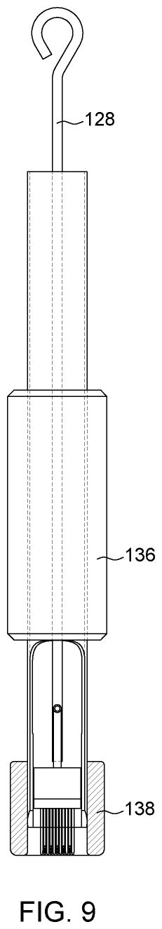

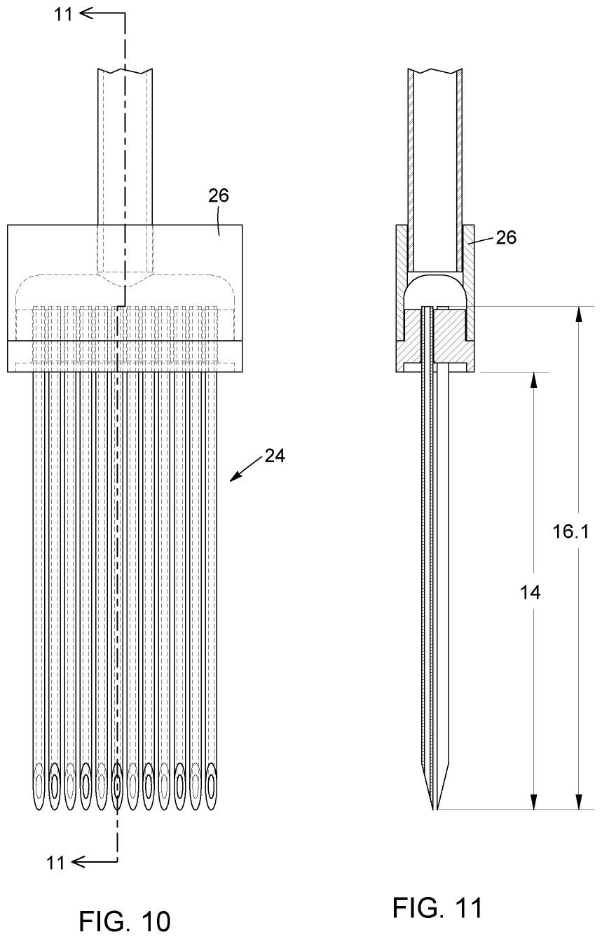

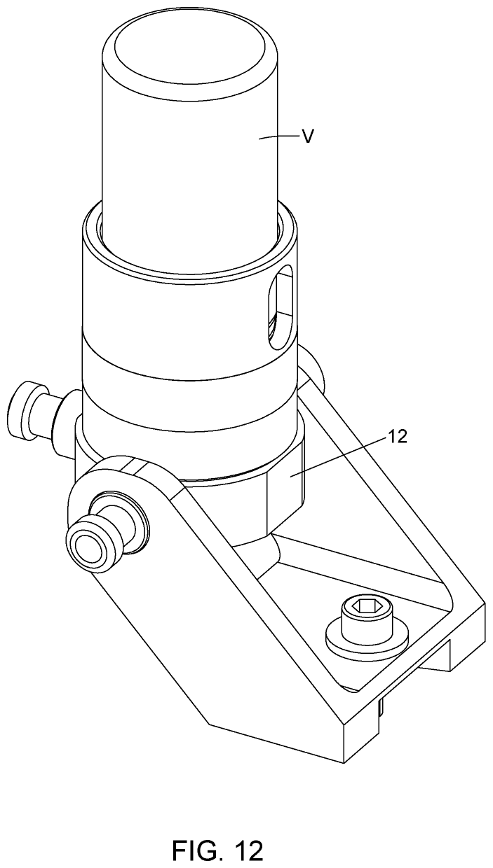

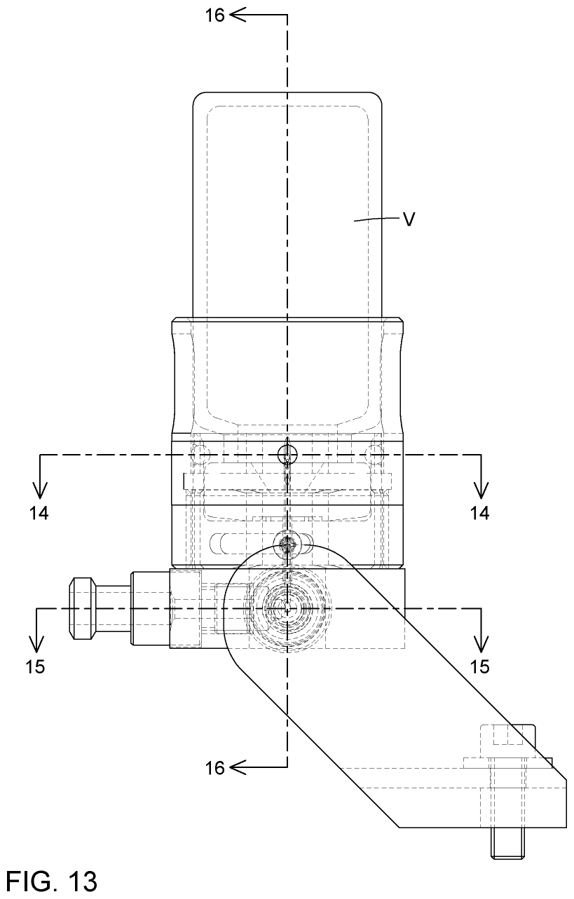

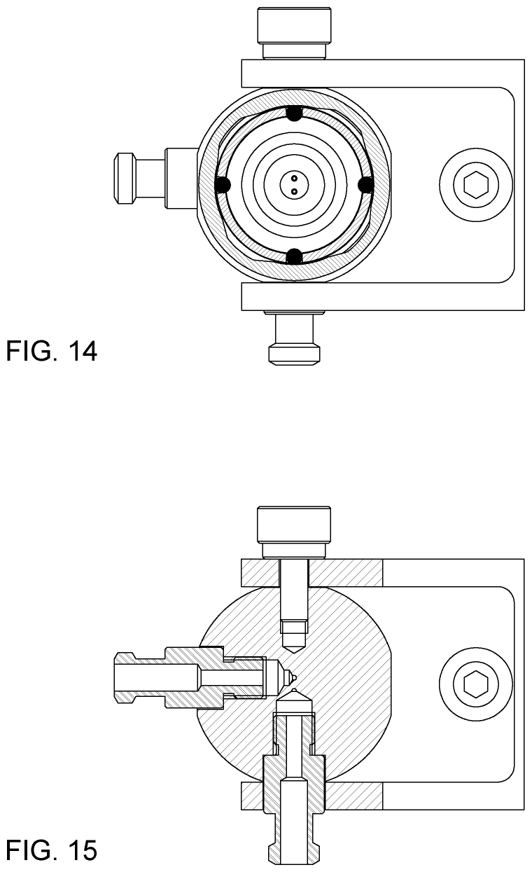

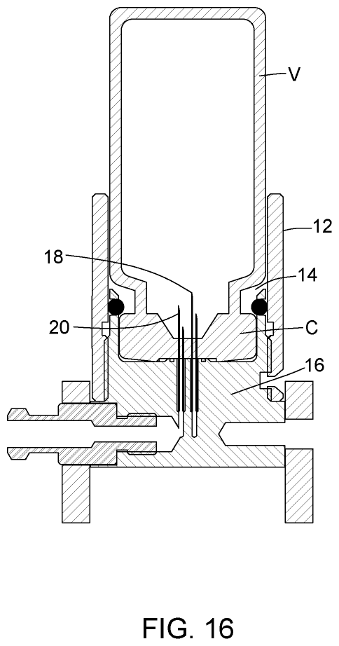

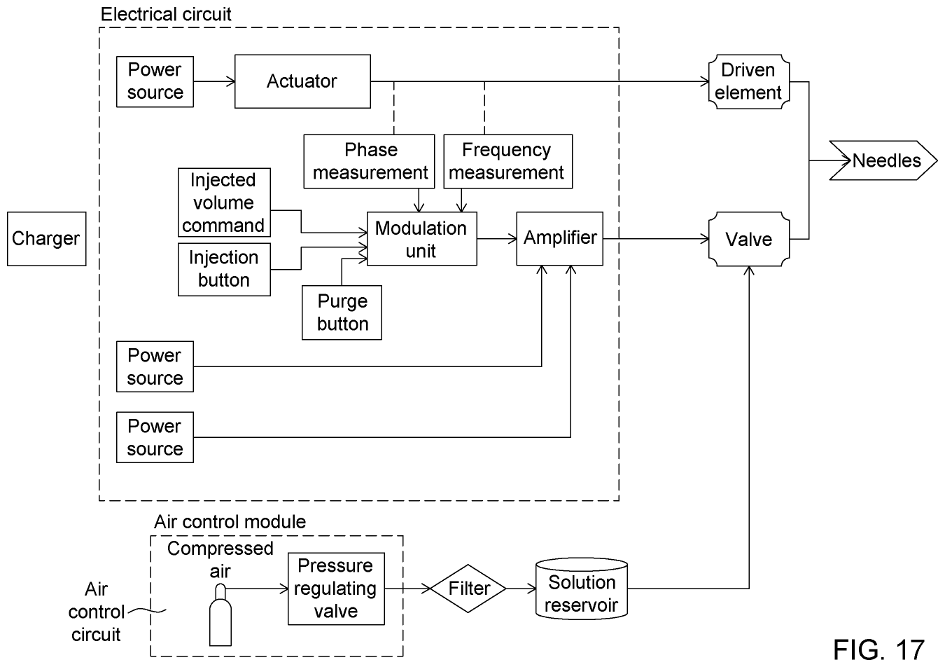

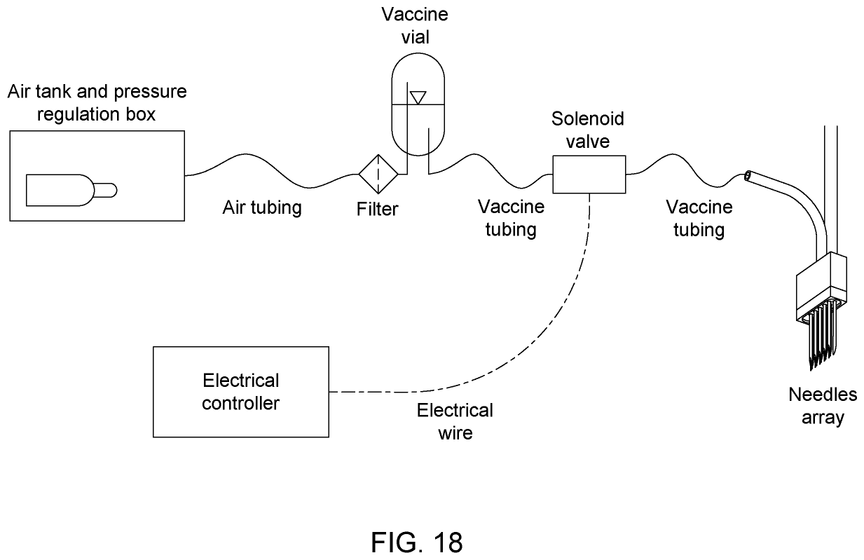

In FIGS. 1 to 5, a device 10 for repeated intradermal injections within an organic tissue according to a first embodiment is shown. In FIGS. 6 to 9, a device 100 for repeated intradermal injections within an organic tissue according to a second embodiment is shown. FIGS. 10 and 11 are enlarged fragmentary views of the injection needles of the device of FIG. 1 or of FIG. 6. FIGS. 12 to 16 show different views of the support of the device of FIG. 1 or of FIG. 6, the support defining an internal housing for receiving a vial containing an aqueous solution and comprising a base with first and second hollow needles for piercing the cap or closure of the vial.

The device 10 comprises a support 12 defining an internal housing 14 for receiving a vial V containing an aqueous solution and having a cap C for closing the vial V. The aqueous solution may be a vaccine, emulsion, colloidal solution, dispersion, or suspension of substances comprising immunogenic components such as proteins, peptides, enzymes, nucleic acids, genes, vectors, nanoparticles, microparticles, (attenuated/killed) viral particles, (attenuated/killed) cells, etc. The terms “solution” or “pressurized solution” used herein in the entire disclosure notably cover “an immunogenic solution” and “pressurized immunogenic solution”.

The support 12 comprises a base 16 comprising first and second hollow needles 18, 20 extending upwardly from the base 16. The first and second hollow needles 18, 20 extend along first and second longitudinal axes and each comprising proximal and distal ends.

When a user inserts the vial V in the housing 14, the cap C abuts against the base 16 and each distal end of the first and second needles 18, 20 pierces the cap C for passing through the cap and being located in the vial V.

The device 10 also comprises a source of pressurized or compressed air in fluid communication with the proximal end of the first hollow needle 18 of the base 16 for injecting pressurized air in the vial V. The term “air” used herein in the entire disclosure includes any suitable medium or means that can be used to pressure the solution such as gas, oxygen or nitrogen.

The device 10 further comprises a tube 22 comprising proximal and distal ends. The proximal end of the tube 22 is in fluid communication with the proximal end of the second hollow needle 20 of the base 16 for allowing pressurized solution to pass through the tube 22 that defines a pressurized solution path.

Moreover, the device 10 has a plurality of hollow injection needles 24, each of the hollow injection needles 24 extending along an injection longitudinal axis and comprising proximal and distal ends. An injection head 26 covers the proximal ends of the hollow injection needles 24. The injection head 26 comprises an inlet in fluid communication with the distal end of the tube 22 and an outlet in fluid communication with the proximal ends of the hollow injection needles 24.

In the drawings, the injection component, including the injection head 26, comprises the plurality of hollow injection needles 24 arranged in two rows. The plurality of hollow injection needles may be hypodermic metal needles of 16 mm in length, embedded in the injection head 26 made for instance of 2 mm of polycarbonate plastic. It is understood that the injection head 26 may comprise one hollow injection needle only. It is also understood that the injection head 26 may comprise a plurality of hollow injection needles that comprise two hollow injection needles or a plurality of hollow injection needles arranged into two linear arrays of two, three, four, five or six needles, resulting in an injection component with four, six, eight, ten or twelve needles, or arranged in circular bundles of two, three, four, six, eight or ten needles, or any other configurations of needles.

The device 10 also comprises a driven element 28 extending along a main longitudinal axis and comprising proximal and distal ends, the distal end of the driven element 28 being connected to the injection head 26. An actuator 30 is connected to the proximal end of the driven element 28.

The device 10 further comprises a valve 32 in fluid communication with the pressurized solution path for controlling a flow of the pressurized solution into the tube 22 from the vial V up to the distal ends of the hollow injection needles 24.

In use, the actuator 30 moves the driven element 28 along the main longitudinal axis at a frequency of between 80 Hz and 150 Hz and between a first position, wherein the distal ends of the hollow injection needles 24 are proximate the organic tissue, and a second position, wherein the distal ends of the hollow injection needles 24 are in the organic tissue at a depth of between 1 mm and 4 mm, and wherein, during movements of the hollow injection needles 24 between the first and second positions, the valve 32 is adapted to allow passage of pressurized solution into the hollow injection needles 24 for injecting the pressurized solution into the organic tissue. It is understood that the expression “during movements of the hollow injection needle(s) between the first and second positions” covers a downward movement, i.e. a movement where the hollow injection needle(s) enter into the tissue, and an upward movement, i.e. a movement where the hollow injection needle(s) exit within the tissue, and it is understood that the valve is adapted to allow passage of the pressurized solution into the hollow injection needle(s) when the needle(s) move downward and/or upward.

The actuator 30 and driven element 28 may include different electrical, mechanical and/or electromechanical components. The device 10 may also include a frame 34 sized and configured for supporting the actuator 30. The actuator 30 is generally secured to the frame. In some embodiments, the actuator 30 is removably attached to the frame. The frame 34 may include a hollow cylindrical portion 36 for housing at least partially receiving the driven element 28.

Broadly, the driven element 28 is configured for oscillating within the hollow cylindrical portion 36 and for moving the hollow injection needles 24. The frame 34 is sized and configured for housing and/or enclosing the different electrical, mechanical and/or electromechanical components of the actuator 30 and/or any other components. In the context of the present description, the expression “frame” is intended to broadly encompass any structure that at least partially encloses or provides a structure for the different components of the device 10, which may include, for example and without being limitative, moving components, as well as static (i.e., immobile) components. The frame can sometimes be referred to as an “open frame”.

The hollow injection needles 24 are movable with respect to the frame 34 and the hollow cylindrical portion 36. As illustrated, the injection longitudinal axis of the hollow injection needles coincides or at least extend in a direction generally parallel than a longitudinal axis of the hollow cylindrical portion. The hollow injection needles 24 are configured for linearly oscillating and/or reciprocation relative thereto.

The frame generally includes an armature bar. The armature bar can be secured to the frame at a pivot point at one extremity and is generally positioned such that it can rotate about the pivot point when a force is applied to the other extremity. As it will be described in greater detail, the force may be applied by the actuator or other electrical component(s) and/or device(s).

The hollow cylindrical portion 36 may be provided with a grip for improving the comfort of a user. In some embodiments, the grip is integrally formed with the hollow cylindrical portion 36. Alternatively, the grip could be separable and/or removable from the hollow cylindrical portion 36. The grip can be useful, in some implementations, to control the positioning of the hollow injection needles with respect to the organic tissue receiving the intradermal injections.

The actuator 30 generally includes at least one electromagnetic coil operatively connected to the frame. In some embodiments, the electromagnetic coil is mounted below the armature bar. The electromagnetic coil includes at least one electrical conductor and may take the shape of a wire in the shape of a coil, spiral or helix. In operation, the electromagnetic coil interacts with electric currents and/or magnetic field. It will be readily understood that the electromagnetic coil is configured such that when a current is passed through the wire the coil, it generates a magnetic field.

In some embodiments, the electromagnetic coil could be formed from any type of conductor, but the electromagnetic coil could be, for example and without being limitative, provided in the form of a solid wire wound around a core or form to create an inductor or electromagnet when an electrical current is applied.

As it will be readily understood, the electromagnetic coil could include any number of loops (“turns”) and generally includes a plurality of turns formed using a broad variety of materials.

The actuator 30 is configured to generate the rotary movement of the armature bar and thus causing the linear translation of the hollow injection needles. More particularly, the actuator allows the hollow injection needles 24 to alternate between a first position (i.e., the hollow injection needles are proximate the organic tissue) and a second injection position (i.e., the hollow injection needles penetrate the organic tissue for injecting the intradermal injections).

The actuator 30 generally includes a spring, which may be provided in the shape of a thin and flexible plate. The spring can be mounted on the armature. Upon a vibrating motion, the spring is configured to deform and contact different electrical contact points of the driven element. In some embodiments, the actuator and/or driven element includes a set of electrical contact points. For example, and without being limitative, a first contact point could be located on the spring, and a second contact point could be located on a screw provided on the frame. As it will be described herein below, the different electrical contact points allow the electrical current to flow through the electromagnetic coils and the armature bar when placed in a close-circuit configuration.

When the spring simultaneously contacts the electrical contact points, a portion of the actuator forms a closed circuit, and an electrical current flow in the electromagnetic coil, hence generating a magnetic field attracting the armature bar. As such, the armature bar linearly moves in a downward direction (i.e., parallel to the force of gravity) towards the electromagnetic coil, which in turn imparts a downward movement to the hollow injection movement, thereby allowing the hollow injection needles to move from their first position to their second position. Movement of the armature bar results in the spring to break the electrical contact with the different electrical contact points of the frame, hence forming an open circuit, which interrupts the electrical current from flowing in the electromagnetic coil, thereby causing an interruption in the generated magnetic field. As a result, an upward linear motion is imparted to the armature part, which in turn translates the hollow injection needles in an upward movement towards the first position.

In some embodiments, a motor or a motor assembly or any other mechanical or electromechanically reciprocating mechanism can be provided to impart the vibrating motion to the spring.

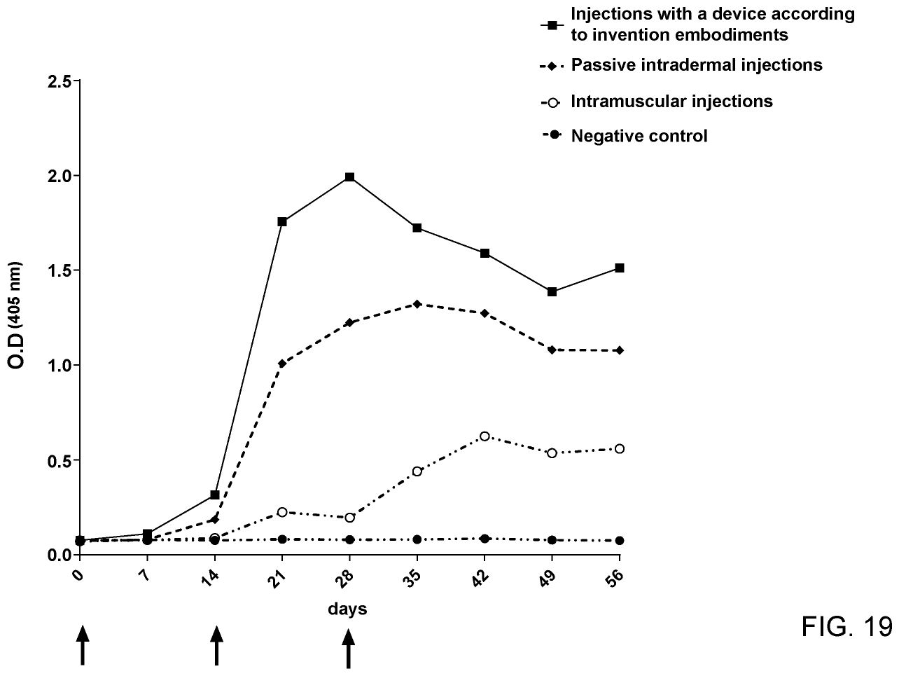

With references to FIGS. 17 and 18, the electrical circuit generally includes the actuator, a measurement unit and a modulation unit. The actuator is the electrical circuit driving the coil. The measurement unit measures the electric waveform sent to the coil by the controller and determines its frequency and its position in time, i.e. its phase. The modulation unit uses the frequency and the phase to generate a control signal that is amplified and fed to the solenoid valve 32 to control the injection of the pressurized solution. In some implementations, the electrical circuit also includes at least one power source for providing power to at least some of the components forming the electrical circuits. It will be readily understood that different operation button(s) and/or command(s) could also be provided for operating and/or controlling the electrical circuit, in turn driving the device 10.

The actuator is operatively connected to the driven element 28. The actuator can include at least one processor. As it will be readily understood, the processor can be implemented as a single unit or as a plurality of interconnected processing sub-units. Also, the processing unit can be embodied by a computer, a microprocessor, a microcontroller, a central processing unit, or by any other type of processing resource or any combination of such processing resources configured to operate collectively as a processing unit. The processor can be implemented in hardware, software, firmware, or any combination thereof, and be connected to the various components of the device system via appropriate communication ports. In some variants, for example and without being limitative, the actuator includes a programmable logic actuator and is remotely connected to the driven element through a wireless network card.

The actuator 30 interfaces with the driven element 28, and as such manages the operation of the driven element. For example, and without being limitative, the actuator can generate an operating signal that is sent towards the driven element 28. In some embodiments, the operating signal is an alternating signal which may define, for example and without being limitative, an oscillating sine wave.

The operating signal has wave properties, which may include but are not limited to phase, frequency, amplitude and the like. Some of the wave properties may be varied and/or controlled through appropriate components and means. In the context of the present description, the expression “phase” refers to a position of a point in time on a waveform cycle and a complete cycle is defined as the interval required for the waveform to return to its initial value. The expression “frequency” herein refers to a number of occurrences of a repeating event per unit of time.

The operating signal is received and interpreted as a command by the driven element 28 (or a component thereof). As such, upon the reception of the operating signal, the driven element 28 can execute a command. For example, and without being limitative, in operation, the driven element 28 can receive the operating signal and in response thereto, engages the armature bar in rotation such that a translational movement is imparted to the hollow injection needles. For instance, the hollow injection needles can oscillate between the first position and the second injection position in response to the operating signal.

In some embodiments, the actuator is provided with a dedicated power source. In one exemplary implementation, the power source is operable to generate a 15 V DC voltage and generates energy corresponding to about 84 Wh. Of course, the values could vary, according to the targeted application.

A measurement unit is operatively connected to the actuator. As illustrated, the measurement unit can be embodied by at least one sub-unit. For example, and without being limitative, the measurement unit could comprise a phase measurement sub-unit and a frequency measurement sub-unit, each being respectively operatively connected to the actuator. It is to be noted that, in the context of the current description, the expression “measurement unit” may encompass the phase measurement sub-unit and a frequency measurement sub-unit. Of course, one will readily understand that other properties of the wave signal may be measured.

The measurement unit is generally provided downstream of the actuator and upstream of the driven element. In some embodiments, the measurement unit measures the operating signal at its output from the actuator.

The measurement unit is configured to generate at least one measurement signal, which is subsequently sent towards the modulation unit.

The measurement unit may include two sub-units, a first one dedicated to phase measurements and a second one dedicated to frequency measurements. In such an embodiment, each sub-unit generates a respective measurement signal. The respective measurement signals, which are representative of the phase and the frequency of the operating signal, can either be simultaneously or sequentially sent towards the modulation unit.

A modulation unit is operatively connected to the measurement unit and to the valve controlling the pressurized solution into the tube from the vial up to the distal end of the hollow injection needles.

The modulation unit is configured to receive the measurement signal(s) and to output a modulated signal which allows controlling the configuration of the valve. For example, the modulated signal can allow the valve to change from a closed configuration (wherein a passage of the pressurized solution is blocked by the valve) to an open configuration (wherein the pressurized solution can flow through the valve), or vice-versa. In the context of the present description, the expression “modulation” refers to a process of modifying and/or varying at least one property of a periodic waveform. For example, the measurement signal(s) representative of the measurements made on the operating signal can be periodic. As such, the modulation unit can take the periodic measurement signal(s) as an input and output a modulated signal. The outputted modulated signal is sent towards the valve.

Upon reception of the modulated signal, a command is executed by the valve. For example, and without being limitative, when predetermined properties of the operating signal are measured (e.g., a predetermined phase and/or frequency), the modulation unit sends a modulated signal for controlling the valve, e.g., opening the valve, such that the pressurized solution can flow therethrough (open configuration), or, alternatively, closing the valve, such that the passage of the pressurized solution is blocked by the valve (closed configuration).

The modulation unit hence allows to synchronize the movement of the hollow injection needles with respect to the frame and the injection of the pressurized solution within the organic tissue. More specifically, the properties of the operating signal to be sent to the driven element 28 can be measured (i.e., characterized), resulting in the measurement signal(s). The measurement signal(s) are then sent to the modulation unit, which in turn generates the modulated signal to be sent to the valve. Such a configuration of the actuator, the measurement unit and the modulation unit allow determining when the hollow injection needles are inserted in the organic tissue, thereby enabling to open the valve and injecting the pressurized solution in within organic tissue.

In some embodiments, the modulation unit is configured to perform pulse-width modulation (PWM) or pulse duration modulation (PDM), or any other signal processing technique(s) and/or method(s) already known by one skilled in the art.

In some embodiments, for example and without being limitative when the modulated signal has a relatively small amplitude, an amplifier could be provided downstream of the modulation unit. As it will be readily understood, the amplifier is an electronic device that can increase the power of a signal.

The amplifier is a two-port electronic component. In some embodiments, each port is associated with a respective power source. For example, a first power source can be associated (i.e., operatively connected) with a first port. For example, and without being limitative, the first power source can be configured to produce 24 V DC voltage at a power greater than 20 W. For example, a second power source can be associated (i.e., operatively connected) with a second port. For example, and without being limitative, the second power source can be configured to produce 3 V DC voltage at a power greater than 2 W. Of course, the design and configuration of the amplifier can vary, according to the needed operating conditions of a targeted application.

The modulation unit can be provided with button(s) and command(s) for operating the same. For example, the electrical circuit can include an injection button operatively connected with the modulation unit to start an injection cycle or, alternatively, to stop an injection cycle. The electrical circuit can also include a purge button associated with the modulation unit. Such a purge button could be useful, for example and without being limitative, purging the tube, the valve and/or the hollow injection needles between different (e.g., subsequent or consecutive) injection cycles.

In one embodiment, the electrical circuit is also provided with an injected volume command operatively connected with the modulation unit. Such a command allows, for example, to predetermine the amount (i.e., the volume) of the pressurized solution to be injected within the organic tissue.

The device 10 may also include a propulsion/compressed/pressurized air/gas/oxygen/nitrogen control module cooperating with the electrical circuit. The propulsion gas control module is in fluid communication with the valve. The propulsion gas control module is provided upstream of a solution reservoir (the vial V) and is configured to provide the pressurized solution to the valve.

The air control module includes an air control circuit. The air control circuit can include a tank of pressurized or compressed air, or any other device to provide compressed air to the valve. The air control circuit includes a pressure regulating valve to adjust and/or control the pressure of the compressed air at the output of the compressed air tank.

In some embodiments, a filter is provided downstream of the air control module and upstream of the solution reservoir. The filter filtrates particles bigger than a predetermined size, which may prevent or at least reduce the contamination of the pressurized solution to be injected within the organic tissue.

The solution reservoir is in fluid communication with the valve, for example and without being limitative through the tube. As such, a certain amount of pressurized solution is present near or at the input of the valve, and upon the opening of the valve by the electrical circuit, the pressurized solution can flow towards the distal ends of the hollow injection needles for the injection within the organic tissue.

As described above, the electrical circuit and the air control module cooperate to control and adjust the amount (i.e., volume) of the pressurized solution to be injected within the organic tissue, and also enables the injection of the pressurized solution within the organic tissue.

The hollow injection needles are mounted with an immunization solution or vaccine flow control system allowing the device 10 to actively inject pressurized solution under the dermis at a rate of 80 to 150 microinjections per second. In short, vaccine administration occurs during accurately timed intervals of a few microseconds.

The device 10 may be portable, i.e. to carry its own independent power source (battery), so that the device can be easily used to vaccinate animals in the field. The added benefit of this device for veterinary use is the possibility for animal identification by mixing the vaccine with temporary or permanent ink, and possibly including additional information such as the vaccine type, dosing and date of vaccination.

The flow intervals can be precisely controlled and timed to occur exactly when the needle distal ends (bevels) are beneath the skin surface, i.e. only when the hollow injection needles are 1.5 to 3.0 millimeters within the dermis.

This device 10 allows for the administration of vaccine preparations at a rate of 0.5 ml to 1.0 ml in 30 to 60 seconds, considerably increasing the volume of vaccine typically delivered by the intradermal route by other devices currently available on the market or in clinical development. This device 10 may significantly improve the efficacy of intradermally-delivered vaccines.

As discussed above, the device 10 also comprises the needle-arrow holder and guide, the vaccine vial holder/support, the solenoid valve and the tubing arrangement.

The injection parameters allowed by the device are a frequency of oscillation between 80 Hz and 150 Hz, preferably approximately 130 Hz, a skin injection at a depth of 1 mm to 4 mm (preferably 2 mm, depending on skin type/thickness), a capacity to inject a volume of 1 ml to humans and 2 to 5 mL to animals, discontinuous liquid injection that can be accurately timed, both in duration and start.

The duration of each injection can be as short as only a few microseconds, repeated and equally spaced in time, with the start of each interval precisely timed after skin penetration. Injections can also occur only at every two or three penetrations, if desired.

The length of the needles may be about 7 mm with bevel tips.

Needle types for human: 31G, hypodermic stainless metal needles, OD: 0.26 mm, ID: 0.12 mm, disposable after use, and for animal: needle range 26G-28G, hypodermic stainless metal needles, reusable after sterilization. 26G: OD: 0.46 mm, ID: 0.26 mm. 27G: OD: 0.41 mm, ID: 0.21 mm. 28G: OD: 0.36 mm, ID: 0.18 mm

The pressure under which the solution (vaccine) is being administered/injected may be between 20 psi and 60 psi.

Reverting to FIGS. 6 to 9, the device 100 is identical to the device 10 to the exception that the device 100 comprises a sleeve or penetration depth guide 138. As for the device 10, the device 100 comprises a driven element 128 extending along a main longitudinal axis and comprising proximal and distal ends. The hollow cylindrical portion 136 at least partially receives the driven element 28. The driven element 128 is configured for oscillating within the hollow cylindrical portion 136 and for moving the hollow injection needles and the needles are movable with respect to the hollow cylindrical portion 136.

The sleeve 138 is adapted to maintain a constant distance between the device 100 and the organic tissue such that the distance between the first position, wherein the distal end of the hollow injection needle is proximate the organic tissue, and the second position, wherein the distal end of the hollow injection needle is within the organic tissue, also remains generally constant.

The sleeve 138 is mounted at the distal end of the driven element 128. The sleeve 138 extends from a top to a bottom peripheral end adapted to contact the organic tissue. In one variant, the sleeve 138 surrounds the hollow injection needles to contain any excess solution that may splash or that may not remain in the organic tissue. In one variant, the sleeve 138 at least partially guides the hollow injection needles during movement between the first and second positions and also aids movement of the device 100 on the organic tissue by generally maintaining the axis of the hollow injection needles perpendicular to the organic tissue.

Each of the devices 10, 100 is to be used to intradermally deliver vaccines to animals or humans. The injection system allows for the vaccine to be administered intradermally at the same time as the needle array is oscillating between injection sites. The number of needles that are part of the array (which could vary between one and twelve needles for instance), along with the needle oscillation will allow for a much larger volume of vaccine to be administered/injected to animals/humans than can currently be administered via other intradermal injection methods. Each of the devices 10, 100 can be operated with single-use disposable needles (human vaccination), or alternatively, with needles easily sterilized in a portable steam sterilizer in the field and re-used (animal vaccination).

An important additional advantage is the possibility to use the devices 10, 100 for the delivery of any immunization vaccine platform, such as nucleic acid-based (DNA, RNA) vaccines, polypeptide (protein)-based, virus-like particles (VLP), viral vector vaccines, cell-based immunization suspensions, attenuated or not, that are resuspended in any regulatory approved solution (and could include adjuvants).

As described above, each of the devices 10, 100 may comprise an array of hollow needles through which the immunization solution (vaccine) is injected. The designed needle array is combined with a dynamic injection system allowing the control of rapid instantaneous injections that are sequenced to occur when the needle is under the skin, thus preventing spillage and loss of the vaccine. Typically, the frequency is a hundred injections per second. The injection circuit allows very small amounts of vaccine to be injected at this frequency. It uses a pressurized solution (vaccine) supply tank/reservoir and a quick responding on/off (solenoid) valve to control flow path of the pressurized solution.

It is possible that vaccine uptake into cells can be accelerated or improved through the inclusion of an electroporation process (short electric pulses to stimulate passage of immunogenic through the membrane of immune system cells competent in presenting antigens) consecutive to the intradermal injection.

Each of the devices 10, 100 allows for intradermal vaccination of animals using a specified volume of vaccine (solution). The mechanism of vaccine injection, the penetration of the oscillating needles, helps to active the immune system, thus resulting in a more robust immune response.

### Example 1

Experiments were carried out in rabbits to compare the efficacy of a prior machine where the vaccine solution is first put on the skin surface and where the oscillating needle array is applied to penetrate the site versus the device according to embodiments of the invention.

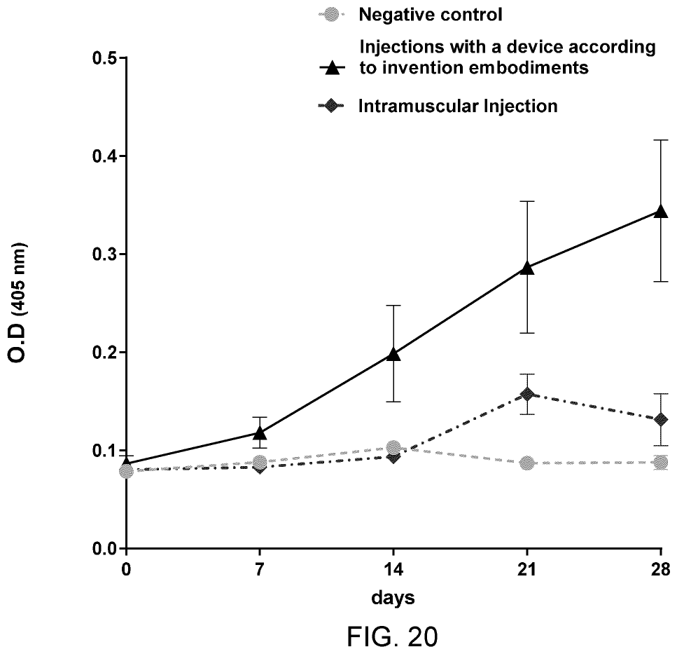

As shown in FIG. 19, the device according to the embodiments of the invention is more efficient at inducing an immune response when compared to other intradermal and intramuscular injections. More particularly, FIG. 19 shows graphs results for Ebola glycoprotein (GP) specific IgG titers that are higher after vaccination with the device of FIG. 1 (full lines with squares) compared to a regular intradermal injection making holes for passive migration of liquid (broken lines with lozenges) with and intramuscular injection (dotted lines with circles). Rabbits (n=3) were immunized (arrows) three times at 2-week intervals with 500 μg of pcDNA3-GP Ebola Zaire or empty pcDNA3 control. The presence of Ebola GP specific IgG in rabbit sera was analyzed after each vaccination by ELISA.

### Example 2

Experiments were carried out in mice to compare the efficacy of the device according to embodiments of the invention vs traditional intramuscular injection (IM) to induce an immune response.

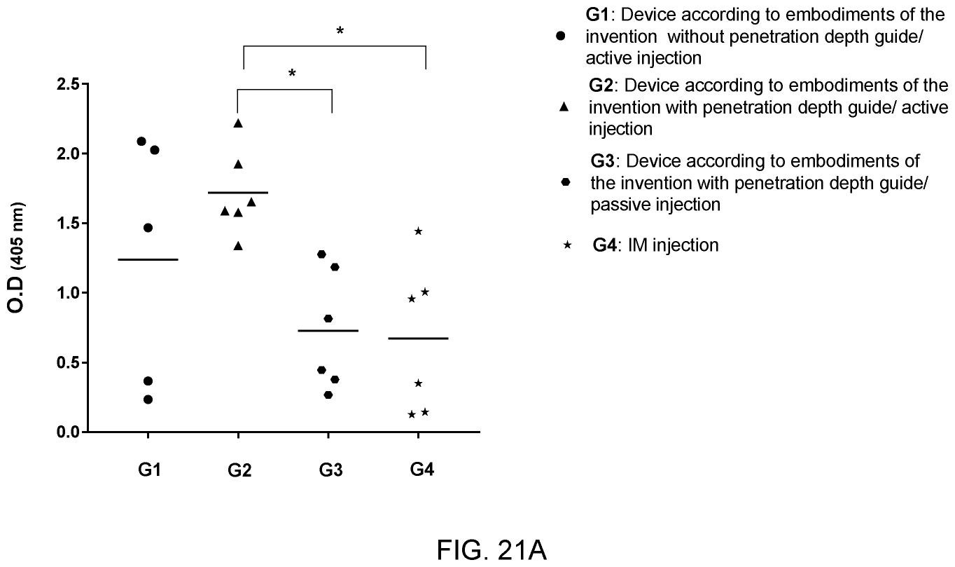

As shown in FIG. 20, each device according to the first and second embodiments is more efficient inducing an immune response when compared to IM injection. Ebola glycoprotein (GP) specific IgG titers are higher after vaccination with the oscillating needle array (full lines with triangles) compared to traditional IM injection (broken lines with rhombuses) and negative control (broken lines with circles). Mice (n=5) were immunized one time with 100 μg of pIDV-II-GP Ebola Zaire or empty pIDV-II plasmid (negative control). The presence of Ebola GP specific IgG in mouse sera was analyzed after vaccination by ELISA.

### Example 3

Experiments were carried out in Guinea pigs to compare the efficacy to induce an immune response of the device according to embodiments of the invention (with or without the sleeve 138 with active or passive injection) vs traditional intramuscular injection (IM).

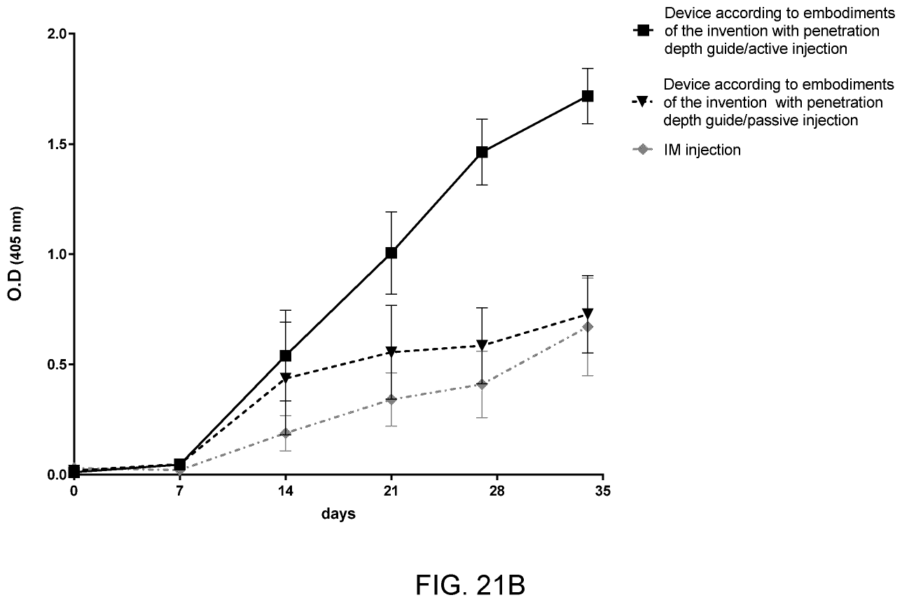

As shown in FIG. 21A, each device according to the first and second embodiments with active injection is more efficient inducing an immune response when compared to IM injection.

More particularly, FIG. 21A shows graphs results for Ebola glycoprotein (GP) specific IgG titers that are higher and less variable after vaccination with the device the sleeve/active injection (G2; triangle dots) compared to the same device without the sleeve/active injection (G1 circular dots), the same device with the sleeve but making holes for passive migration of liquid (G3 hexagon dots, no injection through the needle array and putting the vaccine manually inside the sleeve) or traditional IM injection (G4 star dots).

Guinea pigs (n=6) were immunized one time with 300 μg of pIDV-II-GP Ebola Zaire. The presence of Ebola GP specific IgG in guinea pig sera was analyzed at day 35 after vaccination by ELISA. Each dot represents an animal. Horizontal bar represents the mean of each group. Statistical analysis was made using one-way ANOVA, followed by Tukey multiple comparisons test. *p<0.05.

As shown in FIG. 21B, Ebola GP specific IgG titer were analyzed at different time points after vaccination. The device with the sleeve and active injection (full lines with square) is more efficient inducing an immune response when compared to the same device with passive injection (broken lines with triangles) or IM injection (broken lines with rhombuses).

Guinea pigs (n=6) were immunized one time with 300 μg of pIDV-II-GP Ebola Zaire. The presence of Ebola GP specific IgG in mouse sera was analyzed after vaccination by ELISA

### Example 4

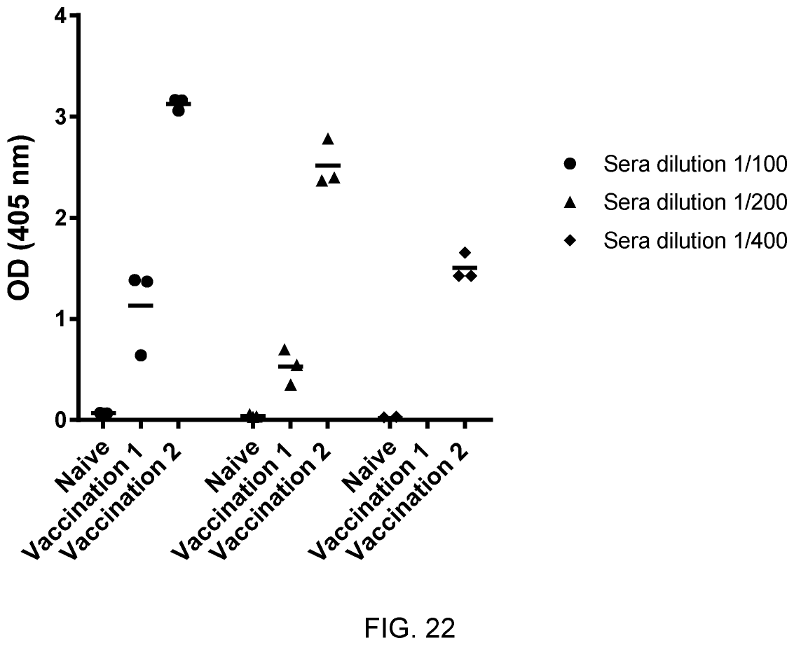

Referring to FIG. 22, experiments were carried out in Non-human primates (NHP) to analyze the efficacy of the device according to first and second embodiments of the invention to induce an immune response against HIV glycoprotein (GP). Animals (n=3) were first vaccinated by IM injection to induce an immune response against HIV GP, followed for a second vaccination (28 days after first vaccination) using the device according to embodiments of the invention. Several dilutions of NHP sera were tested for the presence of HIV GP specific IgG antibodies before (naïve) and after vaccinations by ELISA. Immune responses were dramatically increased after the second vaccination using the device according to the embodiments of the invention. Antibodies titers still remain high after second vaccination at 1/400 sera dilution. Each dot represents an animal. Horizontal bar represents the mean of each group.

The above description of the variants, examples or embodiments should not be interpreted in a limiting manner since other variations, modifications and refinements are possible within the scope of the present invention. Accordingly, it should be understood that various features and aspects of the disclosed variants or embodiments can be combined with or substituted for one another in order to form varying modes of the disclosed invention. The scope of the invention is defined in the appended claims and their equivalents.

1. A device for repeated intradermal injections within an organic tissue, the device comprising:
a support defining an internal housing for receiving a vial containing an aqueous solution and having a cap for closing the vial, the support comprising a base comprising first and second hollow needles extending upwardly from the base, the first and second hollow needles extending along first and second longitudinal axes and each comprising proximal and distal ends, wherein, when a user inserts the vial in the housing, the cap abuts against the base and each distal end of the first and second needles pierces the cap for passing through the cap and being located in the vial;
a source of pressurized air in fluid communication with the proximal end of the first hollow needle of the base for injecting pressurized air in the vial;
a tube comprising proximal and distal ends, the proximal end of the tube being in fluid communication with the proximal end of the second hollow needle of the base for allowing pressurized solution to pass through the tube that defines a pressurized solution path;
a hollow injection needle extending along an injection longitudinal axis, the hollow injection needle comprising proximal and distal ends;
an injection head covering the proximal end of the hollow injection needle; the injection head comprising an inlet in fluid communication with the distal end of the tube and an outlet in fluid communication with the proximal end of the hollow injection needle;
a driven element extending along a main longitudinal axis and comprising proximal and distal ends, the distal end of the driven element being connected to the injection head;
an actuator connected to the proximal end of the driven element; and
a valve in fluid communication with the pressurized solution path for controlling a flow of the pressurized solution into the tube from the vial up to the distal end of the hollow injection needle;
wherein in use, the actuator moves the driven element along the main longitudinal axis at a frequency of between 80 Hz and 150 Hz and between a first position, wherein the distal end of the hollow injection needle is proximate the organic tissue, and a second position, wherein the distal end of the hollow injection needle is within the organic tissue at a depth of between 1 mm and 4 mm; and
wherein, during movements of the hollow injection needle between the first and second positions, the valve is adapted to allow passage of pressurized solution into the hollow injection needle for injecting the pressurized solution into the organic tissue.

2. The device of claim 1, wherein the depth is between 1.5 mm and 3.0 mm.

3. The device of claim 2, wherein the frequency is between 100 Hz and 130 Hz.

4. The device of claim 3, wherein pressure of the pressurized solution is between 20 psi and 60 psi.

5. The device of claim 2 wherein the valve is a solenoid valve.

6. The device of claim 2, wherein the driven element comprises a shaft extending along the main longitudinal axis.

7. The device of claim 1, wherein, in use, the device allows injection of pressurized solution at a rate of 0.5 ml to 1.0 ml in 30 to 60 seconds.

8. The device of claim 7, comprising a filter between the source of pressurized air and the proximal end of the first hollow needle of the base.

9. (canceled)

10. The device of claim 1, wherein the hollow injection needle is a first hollow injection needle and wherein the device comprises at least one additional hollow injection needle.

11. The device of claim 10, wherein the distal end of the driven element comprises a sleeve, the sleeve extending from a top to a bottom peripheral end adapted to contact the organic tissue.

12. The device of claim 11, wherein the sleeve surrounds the hollow injection needles.

13. (canceled)

14. The device of claim 12, wherein, in use, the bottom peripheral end of the sleeve contacts the organic tissue such that the hollow injection needles are generally perpendicular to the organic tissue.

15. A device for repeated intradermal injections within an organic tissue, the device comprising:
a support defining an internal housing for receiving a vial containing an aqueous solution and having a cap for closing the vial, the support comprising a base comprising first and second hollow needles extending upwardly from the base, the first and second hollow needles extending along first and second longitudinal axes and each comprising proximal and distal ends, wherein, when a user inserts the vial in the housing, the cap abuts against the base and each distal end of the first and second needles pierces the cap for passing through the cap and being located in the vial;
a source of pressurized air in fluid communication with the proximal end of the first hollow needle of the base for injecting pressurized air in the vial;
a tube comprising proximal and distal ends, the proximal end of the tube being in fluid communication with the proximal end of the second hollow needle of the base for allowing pressurized solution to pass through the tube that defines a pressurized solution path;
a plurality of hollow injection needles, each of the hollow injection needle extending along an injection longitudinal axis and comprising proximal and distal ends;
an injection head covering the proximal ends of the hollow injection needles; the injection head comprising an inlet in fluid communication with the distal end of the tube and an outlet in fluid communication with the proximal ends of the hollow injection needles;
a driven element extending along a main longitudinal axis and comprising proximal and distal ends, the distal end of the driven element being connected to the injection head;
an actuator connected to the proximal end of the driven element; and
a valve in fluid communication with the pressurized solution path for controlling a flow of the pressurized solution into the tube from the vial up to the distal ends of the hollow injection needles;
the valve is adapted to allow passage of pressurized solution into the hollow injection needles for injecting the pressurized solution into the organic tissue.

16. (canceled)

17. (canceled)

18. (canceled)

19. (canceled)

20. (canceled)

21. The device of claim 15, wherein, in use, the device allows injection of pressurized solution at a rate of 0.5 ml to 1.0 ml in 30 to 60 seconds.

22. The device of claim 21, comprising a filter between the source of pressurized air and the proximal end of the first hollow needle of the base.

23. The device of claim 21, wherein the plurality of hollow injection needles comprises a first array of hollow injection needles and a second array of hollow injection needles.

24. The device of claim 23, wherein the first and second arrays of hollow injection needles are generally parallel and side by side.

25. The device of claim 23, comprising means for measuring remaining quantity of solution or pressurized solution within the vial.

26. The device of claim 15, wherein the distal end of the driven element comprises a sleeve, the sleeve extending from a top to a bottom peripheral end adapted to contact the organic tissue, and wherein the sleeve surrounds the hollow injection needles.

27. The device of claim 26, wherein in use, the actuator moves the driven element along the main longitudinal axis at a frequency of between 80 Hz and 150 Hz and between a first position, wherein the distal ends of the hollow injection needles are proximate the organic tissue, and a second position, wherein the distal ends of the hollow injection needles are in the organic tissue at a depth of between 1 mm and 4 mm; and wherein, during movements of the hollow injection needles between the first and second positions.

28. (canceled)

29. (canceled)
